(19) United States
(12) Patent Application Publication
(10) Pub. No.: US 2021/0177374 A1
(43) Pub. Date: Jun. 17, 2021

(54) BIOMETRIC MEASUREMENT AND QUALITY ASSESSMENT

(71) Applicant: KONINKLIJKE PHILIPS N.V., EINDHOVEN (NL)

(72) Inventors: MARCIN ARKADIUSZ BALICKI, CAMBRIDGE, MA (US); CHRISTINE SWISHER, SAN DIEGO, CA (US)

(21) Appl. No.: 17/269,295

(22) PCT Filed: Aug. 13, 2019
(86) PCT No.: PCT/EP2019/071740

§ 371 Date: Feb. 18, 2021

Related U.S. Application Data
(60) Provisional application No. 62/721,792, filed on Aug. 23, 2018.

(51) Int. Cl. A61B 8/08; A61B 8/14; A61B 8/00; G06N 3/02; G16H 30/40; G16H 50/20; G16H 50/30

(52) U.S. Cl. CPC A61B 8/0866; A61B 8/085; A61B 8/145; A61B 8/463; G16H 50/30; A61B 8/467; G06N 3/02; G16H 30/40; G16H 50/20; A61B 8/5223

(57) ABSTRACT

The present disclosure describes imaging systems configured to determine the accuracy of anatomical measurements obtained from image data. Systems may include an ultrasound transducer configured to acquire echo signals responsive to ultrasound pulses transmitted toward a target region. Systems can also include a graphical user interface configured to display a biometry tool widget, such as a caliper, for acquiring a measurement of an anatomical feature within the target region from at least one image frame generated from the ultrasound echoes. Systems can also include one or more processors configured to determine a confidence metric indicative of the accuracy of the measurement. The processors can also be configured to cause the graphical user interface to display a graphical indicator corresponding to the confidence metric. The processors can implement one or more neural networks, and can derive additional information, such as gestational age or weight, from the anatomical measurements acquired.

### TECHNICAL FIELD

The present disclosure pertains to ultrasound systems and methods for measuring anatomical features via ultrasound imaging and determining the associated measurement quality using at least one neural network. Particular implementations involve systems configured to generate probability-based confidence levels for each measurement obtained via an ultrasound imaging system equipped with one or more biometry tools.

### BACKGROUND

Ultrasound fetal biometry is commonly used to estimate fetal age and growth trajectories for pregnancy management, and is the primary diagnostic tool for potential fetal health defects. Fetal disorders are often identified based on discrepancies between the actual and expected relationships of certain anatomical measurements at a given gestational age. The accuracy of fetal disorder identification is highly dependent on a sonographer's skill in ultrasound image acquisition and measurement extraction, which may require pinpointing the correct imaging plane for a particular anatomical measurement and using virtual instruments, e.g., a caliper, to obtain the measurement. Intra- and inter-observer variability of ultrasound imaging and assessment frequently contributes to incorrect estimation of fetal size and growth, which leads to unnecessary repeat examinations, increased cost, and unwarranted stress for expecting parents. Moreover, a shortage of trained sonographers and added ultrasound exam prescriptions increase the pressure on healthcare providers to use less trained sonographers, and to reduce the number of repeat examinations and the length of each patient stay. Accordingly, new technologies configured to obtain and analyze ultrasound images of a fetus or other anatomical features with improved accuracy and consistency in less time are needed.

### SUMMARY

The present disclosure describes systems and methods for obtaining and analyzing ultrasound images of various anatomical objects by employing at least one deep learning neural network. While examples herein specifically address prenatal evaluations of a fetus, it should be understood to those skilled in the art that the disclosed systems and methods are described with respect to fetal assessment for illustrative purposes only, and that anatomical measurements can be performed at a range of timepoints on a variety of objects within a patient, including but not limited to the heart and lungs, for instance. In some embodiments, the system may be configured to improve the accuracy, efficiency and automation of prenatal ultrasound scans, or ultrasound scanning protocols associated with other clinical applications (e.g., cardiac, liver, breast, etc.). The systems may reduce ultrasound examination errors by determining the quality of the obtained measurements in view of the current measurement set as a whole, prior measurements of the anatomical feature and/or patient, and known health risks. Example systems implement a deep learning approach to generate a probability that each measurement obtained from the ultrasound data belongs to a unique set, thereby providing a confidence metric that may be displayed to a user. In some embodiments, the neural network can be trained using expert data interpreting a wide range of relationships between anatomical measurements and natural population variability. The results can be used to guide a user, e.g., a sonographer, to redo a particular measurement.

In accordance with some examples of the present disclosure, an ultrasound imaging system may include an ultrasound transducer configured to acquire echo signals responsive to ultrasound pulses transmitted toward a target region. The system may also include a graphical user interface configured to display a biometry tool widget for acquiring a measurement of an anatomical feature within the target region from at least one image frame generated from the ultrasound echoes. The system can also include one or more processors in communication with the ultrasound transducer and configured to determine a confidence metric indicative of an accuracy of the measurement, and cause the graphical user interface to display a graphical indicator corresponding to the confidence metric.

In some examples, the processors are configured to determine the confidence metric by inputting the at least one image frame into a first neural network trained with imaging data comprising the anatomical feature. In some embodiments, the processors are further configured to determine the confidence metric by inputting a patient statistic, a prior measurement of the anatomical feature, a derived measurement based on the prior measurement, a probability that the image frame contains an anatomical landmark associated with the anatomical feature, a quality level of the image frame, a setting of the ultrasound transducer, or combinations thereof, into the first neural network. In some examples, the probability that the image frame contains the anatomical landmark indicates whether a correct imaging plane has been obtained for measuring the anatomical feature. In some embodiments, an indication of the probability that the image frame contains the anatomical landmark is displayed on the graphical user interface. In some examples, the derived measurement comprises a gestational age or an age-adjusted risk of a chromosomal abnormality. In some embodiments, the patient statistic comprises a maternal age, a patient weight, a patient height, or combinations thereof. In some examples, the quality level of the image frame is based on a distance of the anatomical feature from the ultrasound transducer, an orientation of the biometry tool widget relative to the ultrasound transducer, a distance of a beam focus region to the anatomical feature, a noise estimate obtained via frequency analysis, or combinations thereof. In some examples, the graphical user interface is not physically coupled to the ultrasound transducer.

In some embodiments, the processors are further configured to apply a threshold to the confidence metric to determine whether the measurement should be re-acquired, and cause the graphical user interface to display an indication of whether measurement should be re-acquired. In some examples, the biometry tool widget comprises a caliper, a trace tool, an ellipse tool, a curve tool, an area tool, a volume tool, or combinations thereof. In some examples, the anatomical feature is a feature associated with a fetus or a uterus. In some embodiments, the processors are further configured to determine a gestational age and/or a weight estimate based on the measurement. In some examples, the first neural network comprises a multilayer perceptron network configured to perform supervised learning with stochastic dropout, or an autoencoder network configured to generate a compressed representation of the image frame and the measurement, and compare the compressed representation to a manifold of population-based data.

In accordance with some examples of the present disclosure, a method of ultrasound imaging can involve acquiring echo signals responsive to ultrasound pulses transmitted into a target region by a transducer operatively coupled to an ultrasound system. The method can also involve displaying a biometry tool widget for acquiring a measurement of an anatomical feature within the target region from at least one image frame generated from the ultrasound echoes. The method can further involve determining a confidence metric indicative of an accuracy of the measurement, and causing the graphical user interface to display a graphical indicator corresponding to the confidence metric.

In some examples, determining the confidence metric comprises inputting the at least one image frame into a first neural network trained with imaging data comprising the anatomical feature. In some embodiments, the method may further involve inputting a patient statistic, a prior measurement of the anatomical feature, a derived measurement based on the prior measurement, a probability that the image frame contains an anatomical landmark associated with the anatomical feature, a quality level of the image frame, a setting of the ultrasound transducer, or combinations thereof, into the first neural network. In some embodiments, the patient statistic comprises a maternal age, a patient weight, a patient height, or combinations thereof. In some examples, the derived measurement comprises a gestational age or an age-adjusted risk of a chromosomal abnormality. In some embodiments, the method may further involve determining a gestational age and/or a weight estimate based on the measurement.

Any of the methods described herein, or steps thereof, may be embodied in non-transitory computer-readable medium comprising executable instructions, which when executed may cause a processor of a medical imaging system to perform the method or steps embodied herein.

### BRIEF DESCRIPTION OF THE DRAWINGS

FIG. 1 is a block diagram of an ultrasound system in accordance with principles of the present disclosure.

FIG. 2 is a block diagram of an operational arrangement of system components implemented in accordance with principles of the present disclosure.

FIG. 3 is a diagram of an autoencoder network implemented in accordance with principles of the present disclosure.

FIG. 4 is a diagram showing additional components of the ultrasound system of FIG. 1.

FIG. 5 is a flow diagram of a method of ultrasound imaging performed in accordance with principles of the present disclosure.

### DETAILED DESCRIPTION

The following description of certain embodiments is merely exemplary in nature and is in no way intended to limit the invention or its applications or uses. In the following detailed description of embodiments of the present systems and methods, reference is made to the accompanying drawings which form a part hereof, and which are shown by way of illustration specific embodiments in which the described systems and methods may be practiced. These embodiments are described in sufficient detail to enable those skilled in the art to practice presently disclosed systems and methods, and it is to be understood that other embodiments may be utilized and that structural and logical changes may be made without departing from the spirit and scope of the present system. Moreover, for the purpose of clarity, detailed descriptions of certain features will not be discussed when they would be apparent to those with skill in the art so as not to obscure the description of the present system. The following detailed description is therefore not to be taken in a limiting sense, and the scope of the present system is defined only by the appended claims.

Fetal size and growth trajectories are important indicators of fetal health. For example, fetal growth disorders are often identified based on discrepancies between the actual and expected biometric measurements for a given gestational age. Due in part to frequent human error, such discrepancies are often attributed to inaccurate anatomical measurements obtained via ultrasound imaging, leading to false positive results. Likewise, measurement error can fail to uncover a real discrepancy, leading to false negatives. Accordingly, determining which measurements are accurate and which are not is crucial to accurate anatomical assessment of a fetus or additional anatomical features of a patient. Systems and methods herein can improve ultrasound image acquisition and assessment technology configured to measure various anatomical features by distinguishing between accurate and inaccurate anatomical measurements, and/or by reducing or eliminating the acquisition of inaccurate measurements. Systems herein can be configured to quantify the accuracy of a particular measurement by determining a confidence level for the measurement. Particular implementations involve acquiring ultrasound images of a fetus and obtaining various anatomical measurements therefrom. Based on the obtained measurements, one or more derived measurements, such as gestational age, fetal weight and/or the presence of an anatomical abnormality can be determined. By determining the confidence level associated with the obtained measurements, systems herein may reduce the misinterpretation of fetal images, thereby reducing the likelihood of false positives and false negatives with respect to abnormality detection, and improving the accuracy of population-based growth comparisons, for example. Specific implementations can be configured to improve the confidence level associated with obtained anatomical measurements by identifying the correct imaging planes needed to acquire each measurement. Image quality may also be improved by enhanced acoustic coupling and automatic selection of the optimal image settings necessary to acquire a specific image. Such improvements may be especially drastic when examining difficult-to-image patients, e.g., obese patients.

An ultrasound system according to the present disclosure may utilize a neural network, for example a deep neural network (DNN), a convolutional neural network (CNN), a recurrent neural network (RNN), an autoencoder neural network, or the like, to determine the quality of a measurement of an anatomical feature. In some examples, a neural network may also be employed to determine the quality of an ultrasound image from which the measurement is initially obtained. Image quality can encompass whether the image is visually easy or difficult to interpret, or whether the image includes certain prerequisite landmark features necessary to acquire accurate and consistent measurements of a specific target feature. In various examples, the neural network(s) may be trained using any of a variety of currently known or later developed learning techniques to obtain a neural network (e.g., a trained algorithm or hardware-based system of nodes) that is configured to analyze input data in the form of ultrasound image frames, measurements, and/or statistics and determine the quality of the measurements, which may be embodied in a confidence level output by the network for each measurement.

An ultrasound system in accordance with principles of the present invention may include or be operatively coupled to an ultrasound transducer configured to transmit ultrasound pulses toward a medium, e.g., a human body or specific portions thereof, and generate echo signals responsive to the ultrasound pulses. The ultrasound system may include a beamformer configured to perform transmit and/or receive beamforming, and a display configured to display, in some examples, ultrasound images generated by the ultrasound imaging system. The ultrasound imaging system may include one or more processors and at least one model of a neural network, which may be implemented in hardware and/or software components. The neural network can be trained to evaluate the accuracy of anatomical measurements obtained via a biometry tool. In some examples, one or additional neural networks can be trained to evaluate the quality and content sufficiency of the images used to obtain the measurements. The neural networks can be communicatively coupled or integrated into one multi-layered network.

The neural network implemented according to the present disclosure may be hardware—(e.g., neurons are represented by physical components) or software-based (e.g., neurons and pathways implemented in a software application), and can use a variety of topologies and learning algorithms for training the neural network to produce the desired output. For example, a software-based neural network may be implemented using a processor (e.g., single or multi-core CPU, a single GPU or GPU cluster, or multiple processors arranged for parallel-processing) configured to execute instructions, which may be stored in computer readable medium, and which when executed cause the processor to perform a trained algorithm for evaluating image and/or measurement quality. The ultrasound system may include a display or graphics processor, which is operable to arrange the ultrasound images (2D, 3D, 4D etc.) and/or additional graphical information, which may include annotations, confidence metrics, user instructions, tissue information, patient information, indicators, color coding, highlights, and other graphical components, in a display window for display on a user interface of the ultrasound system. In some embodiments, the ultrasound images and associated measurements may be provided to a storage and/or memory device, such as a picture archiving and communication system (PACS) for post-exam review, reporting purposes, or future training (e.g., to continue to enhance the performance of the neural network), especially the images used to produce measurements associated with high confidence levels. The display can be remotely located, and interacted with by users other than the sonographer conducting the imaging, in real-time or asynchronously. In some examples, ultrasound images and/or associated measurements obtained during a scan may not be displayed to the user operating the ultrasound system, but may be analyzed by the system for the presence or absence of potential anatomical abnormalities or measurement errors as an ultrasound scan is performed. According to such implementations, the images and/or measurements may be distilled in a report generated for review by a user, such as a sonographer, obstetrician, or clinician.

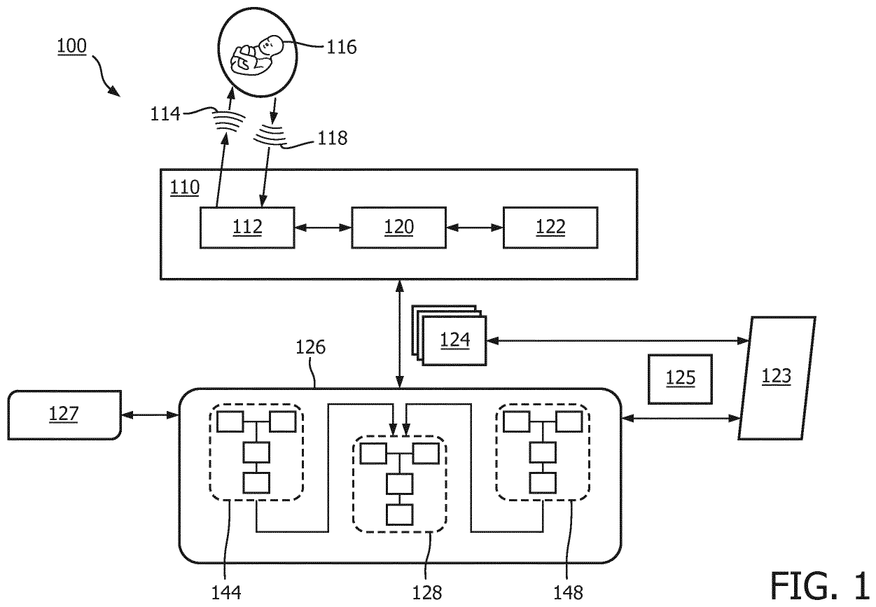

FIG. 1 shows an example ultrasound system according to principles of the present disclosure. The ultrasound system 100 may include an ultrasound data acquisition unit 110. The ultrasound data acquisition unit 110 can include an ultrasound probe which includes an ultrasound sensor array 112 configured to transmit ultrasound pulses 114 into a region 116 of a subject, e.g., abdomen, and receive ultrasound echoes 118 responsive to the transmitted pulses. The region 116 may include a developing fetus, as shown, or a variety of other anatomical objects, such as the heart or the lungs. As further shown, the ultrasound data acquisition unit 110 can include a beamformer 120 and a signal processor 122, which can be configured to generate a stream of discrete ultrasound image frames 124 from the ultrasound echoes 118 received at the array 112. In addition, one or more image biometry tool widgets 123, e.g., a caliper, trace tool and/or an ellipse tool, curve tool, area tool, volume tool, etc., can be configured to obtain one or more measurements 125 of an anatomical feature visible within image frame 124. The tool widget measurements may be manual requiring user input, or autonomous. The image frames 124 and/or associated measurements 125 can be communicated to a data processor 126, e.g., a computational module or circuitry, configured to determine the accuracy of the measurements. In some examples, the data processor 126 may be configured to determine measurement accuracy by implementing at least one neural network, such as neural network 128, which can be trained to estimate the accuracy of measurements obtained from the ultrasound images. The data processor 126 may also be configured to implement an image classification network 144 and/or an image quality network 148, the outputs of which may be input into neural network 128 in some embodiments to improve the accuracy of the network 128. In various examples, the data processor 126 can also be coupled, communicatively or otherwise, to a database 127 configured to store various data types, including training data and newly acquired, patient-specific data.

The ultrasound data acquisition unit 110 can be configured to acquire ultrasound data from one or more regions of interest 116, which may include a fetus, a uterus, and features thereof. The ultrasound sensor array 112 may include at least one transducer array configured to transmit and receive ultrasonic energy. The settings of the ultrasound sensor array 112 can be preset for performing a prenatal scan of a fetus, and in embodiments, can be adjustable during a particular scan. A variety of transducer arrays may be used, e.g., linear arrays, convex arrays, or phased arrays. The number and arrangement of transducer elements included in the sensor array 112 may vary in different examples. For instance, the ultrasound sensor array 112 may include a 1D or 2D array of transducer elements, corresponding to linear array and matrix array probes, respectively. The 2D matrix arrays may be configured to scan electronically in both the elevational and azimuth dimensions (via phased array beamforming) for 2D or 3D imaging. In addition to B-mode imaging, imaging modalities implemented according to the disclosures herein can also include shear-wave and/or Doppler, for example. A variety of users may handle and operate the ultrasound data acquisition unit 110 to perform the methods described herein. In some examples, the user may be an inexperienced, novice ultrasound operator unable to accurately identify each anatomical feature of a fetus required in a given scan. In some cases, the data acquisition unit 110 is controlled by a robot (positioning, settings, etc.), and can replace the human operator data to perform the methods described herein. For instance, the data acquisition unit 110 may be configured to utilize the findings obtained by the data processor 126 to refine one or more image planes and or anatomical measurements obtained therefrom. According to such examples, the data acquisition unit 110 can be configured to operate in automated fashion by adjusting one or more parameters of the transducer, signal processor, or beamformer in response to feedback received from the data processor.

The data acquisition unit 110 may also include a beamformer 120, e.g., comprising a microbeamformer or a combination of a microbeamformer and a main beamformer, coupled to the ultrasound sensor array 112. The beamformer 120 may control the transmission of ultrasonic energy, for example by forming ultrasonic pulses into focused beams. The beamformer 120 may also be configured to control the reception of ultrasound signals such that discernable image data may be produced and processed with the aid of other system components. The role of the beamformer 120 may vary in different ultrasound probe varieties. In some embodiments, the beamformer 120 may comprise two separate beamformers: a transmit beamformer configured to receive and process pulsed sequences of ultrasonic energy for transmission into a subject, and a separate receive beamformer configured to amplify, delay and/or sum received ultrasound echo signals. In some embodiments, the beamformer 120 may include a microbeamformer operating on groups of sensor elements for bother transmit and receive beamforming, coupled to a main beamformer which operates on the group inputs and outputs for both transmit and receive beamforming, respectively.

The signal processor 122 may be communicatively, operatively and/or physically coupled with the sensor array 112 and/or the beamformer 120. In the example shown in FIG. 1, the signal processor 122 is included as an integral component of the data acquisition unit 110, but in other examples, the signal processor 122 may be a separate component. In some examples, the signal processor may be housed together with the sensor array 112 or it may be physically separate from but communicatively (e.g., via a wired or wireless connection) coupled thereto. The signal processor 122 may be configured to receive unfiltered and disorganized ultrasound data embodying the ultrasound echoes 118 received at the sensor array 112. From this data, the signal processor 122 may continuously generate a plurality of ultrasound image frames 124 as a user scans the fetal region 116.

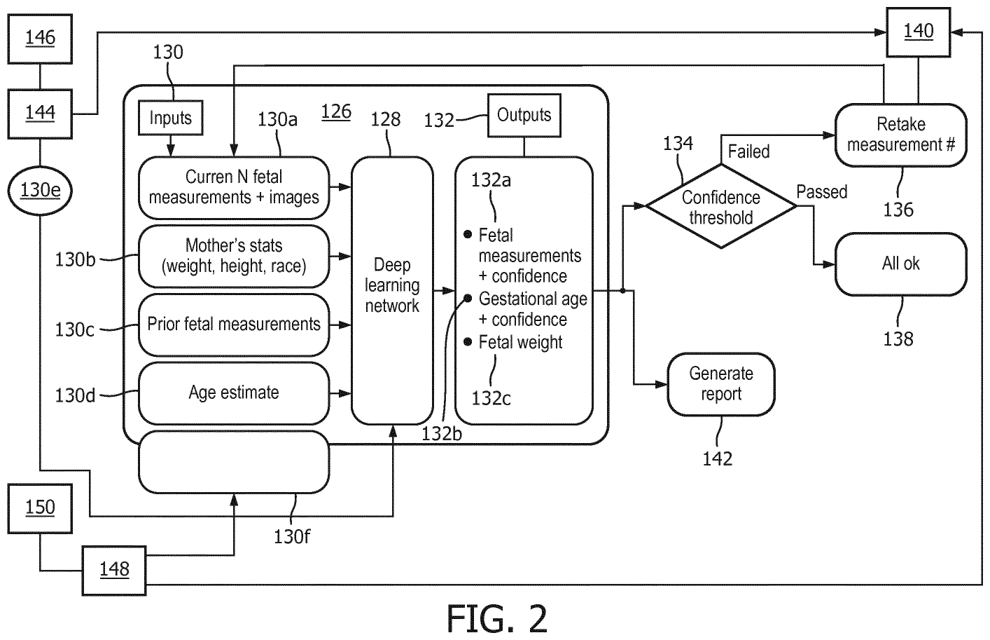

In particular embodiments, neural network 128 may comprise a deep learning network trained, via imaging data, to generate a probability that each measurement 125 obtained via biometry tool widget 123 belongs to a unique set of measurements. An associated confidence level, based on or equal to this probability, is then generated for each measurement, providing a user with a real-time evaluation of measurement accuracy and in some examples, indicating whether one or more measurements should be re-acquired. As explained below with respect to FIG. 2, neural network 128 may process a plurality of distinct inputs, including the outputs generated by the image classification network 144 and the image quality network 148.

FIG. 2 shows an example operational arrangement of components implemented in accordance with system 100, including the various inputs and outputs that can be received and generated, respectively, by the data processor 126. As shown, the data processor 126 can be configured to implement a neural network 128 which can be configured to receive one or more inputs 130. The inputs 130 may vary. For example, the inputs 130 may include current fetal measurements and the corresponding ultrasound images 130a obtained in substantially real time during an ultrasound examination. Fetal measurements obtained via system 100 can include but are not limited to: crown-rump length, head circumference, biparietal diameter, gestational sac diameter, occipitofrontal diameter, femur length, humeral length, abdominal circumference, interocular distance, and/or binocular distance; as well as functional imaging like heart rate, cardiac volume, and/or fetal motion. Additional measurements of other anatomical features, for example a cross-sectional diameter of organs such as the heart or lungs, can also be acquired in some examples, along with additional parameters, such as the angle between two measurements. The inputs may also include various statistics 130b of the mother, including the mother's weight, height, age, race, etc. Prior fetal measurements 130c of a given fetus can also be input. The inputs 130 can further include one or more derived measurements, such as a gestational age estimate 130d, that are based on the direct fetal measurements, e.g., femur length. In some embodiments, derived measurements can include ultrasound markers indicative of increased age-adjusted risk of an underlying fetal aneuploidic or non-chromosomal abnormality. One or more of the aforementioned inputs 130 can be received by neural network 128, which is configured to analyze the inputs 130 and generate one or more outputs 132 based on the inputs. Such outputs 132 can include one or more fetal measurements and an associated confidence level 132a for each measurement, a gestational age estimate and the associated confidence level 132b, and a fetal weight estimate 132c.

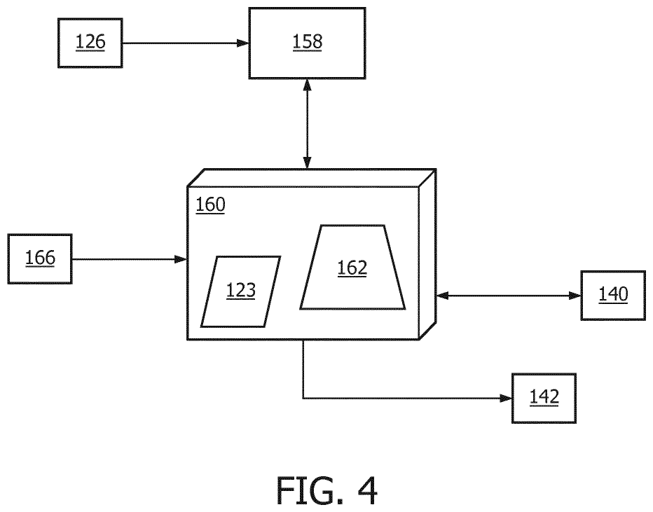

To each output 132 of the neural network 128, a confidence threshold 134 may be applied by the data processor 126 to determine whether the quality of a given measurement is satisfactory, or whether re-measurement is necessary. The thresholds can be tuned empirically, or set directly by the user or reviewer. According to some embodiments, the thresholding result may be conveyed in the form of one or more notifications 140. For example, the data processor 126 can be configured to generate a “Retake Measurement” notification 136 for measurements that do not satisfy the threshold, and an “All OK” notification 138 for measurements that do satisfy the threshold 134. The specific manner in which the notifications are conveyed may vary. For instance, the notifications can include displayed text, as described below with reference to FIG. 4, or various visual and/or audio cues presented to a user. In addition or alternatively, data processor 126 can be configured to generate a report 142, which may include all or select measurements and associated confidence levels determined by neural network 128. An example report 142 may include outputs 132a, 132b and/or 132c obtained during a given scan and any notifications associated therewith.

In some examples, the system 100 can be configured to implement a second neural network to further improve the accuracy of image acquisition and assessment by evaluating the ultrasound probe position relative to the target anatomy, which may be performed prior to implementation of neural network 128. In particular, an image classification network 144, which may comprise a CNN, can be trained to determine whether a given ultrasound image contains the requisite anatomical landmarks for obtaining a particular measurement. For example, biparietal diameter and head circumference measurements may be erroneous if the transthalamic view is not obtained with the ultrasound probe. In the transthalamic view, the thalami and cavum septum pellucideum should both be visible. Similarly, the abdominal circumference measurement may be erroneous if the stomach, umbilical vein, and two ribs on each side of the abdomen are not visible. Accordingly, when biparietal diameter and head circumference measurements are sought, the image classification network 144 can be configured to determine whether the thalami and cavum septum pellucideum are included in a current image frame. Likewise, when the abdominal circumference is sought, the image classification network 144 can be configured to determine whether the stomach, umbilical vein, and two ribs on each side of the abdomen are included in a current image frame. By confirming the presence of one or more landmarks, the image classification network 144 may confirm that the correct imaging plane for a specified anatomical measurement has been obtained, which allows the biometry tool(s) 123 to measure the target feature included in the image with greater confidence. In some examples, a segmentation processor may be implemented in addition to or instead of the image classification network 144 to perform automated segmentation of the target region 116.

The input 146 processed by the image classification network 144 can include the area of the image that is inside a pre-selected circumference. By limiting the area to a pre-selected circumference, the total area searched for one or more anatomical landmarks by the image classification network 144 is reduced, thereby reducing the amount of required data used to train the network and further enhancing the processing efficiency of system 100.

In various embodiments, the output 130e of the image classification network 144 can be utilized as another input source processed by neural network 128. The output 130e can include the numerical probability that a given image frame contains the landmark anatomical features associated with a particular anatomical measurement, thus providing an additional measure of confidence used to evaluate the quality of a particular image and the measurements obtained therefrom. For instance, if the image classification network 144 is not implemented to filter the initial image frames, the likelihood of the final confidence level output(s) 132 being accurate may be reduced because a measurement that is consistent with the population-wide average, but obtained from a suboptimal image plane, may be inaccurate. In some embodiments, the output 130e can be displayed for immediate assessment by the user performing the ultrasound scan, thereby enabling the user to decipher whether the current probe position is adequate for obtaining a given measurement, or whether an adjustment in probe position, orientation and/or settings is needed. For example, a questionable anatomical measurement can be displayed, along with the corresponding image, to the user with a notification 140 that the image may or does lack one or more anatomical landmarks. By providing such an indication to the user before implementation of neural network 128, futile processing steps may be avoided, thereby improving the processing efficiency of system 100.

In additional or alternative embodiments, the system 100 can be configured to implement a third (or second) neural network to further improve the accuracy of image acquisition and assessment by evaluating the quality of ultrasound images obtained during a given scan. In particular, an image quality network 148 can be trained to determine whether a given ultrasound image is of high, low or medium quality. The inputs 150 received by the image quality network 148 can include ultrasound images and/or image settings, such as frequency, gain, etc. Inputs 150 can also include an aberration estimate that degrades image quality, the minimum, maximum and average distance of the measurement from the ultrasound transducer, the orientation of measurement widgets relative to the transducer, the distance of the measurement end-points to the beam focus region, the estimated image resolution along the measurement axis, and/or a noise estimate obtained via frequency analysis in the region surrounding the end-points of the caliper selection, for example. The image quality network 148 can be trained with a plurality of images, each image correlated with the aforementioned inputs 150 and labeled as having high, low or medium quality.

In various examples, the output 130f of the image quality network 144 can be utilized as another input source processed by neural network 128. The output 130f can comprise a numerical probability that a particular image used to obtain a measurement is of requisite quality. In some examples, the output 130f can be generated and used in substantially real time during measurement acquisition in order to provide the user with an early indication of potential measurement errors. For example, upon selecting two measurement end-points, a notification 140 of image quality, e.g., 50%, 75% or 99%, may be generated and displayed, the image quality conveying the likelihood that a particular measurement can be accurately determined, such that a 10% image quality metric would convey a low likelihood that a measurement could be accurately determined based on the image, while a 99% image quality metric would convey a high likelihood that a measurement could be accurately determined based on the image. In some examples, the image quality network 148 can be configured to allow a user to “back into” the inputs 150 to determine which particular input(s) contributed the most to a particular image quality metric.

Neural network 128 can be configured to identify erroneous measurements, or measurements likely to be erroneous, by implementing various supervised or unsupervised learning techniques configured specifically for the anatomical measurement applications described herein. Depending on the techniques employed, the architecture of the neural network may also vary. For example, neural network 128 may comprise a multilayer perceptron (MLP) network configured to perform supervised learning with stochastic dropout. While the specific architecture may vary, the MLP network may generally comprise an input layer, an output layer, and multiple hidden layers. Every neuron within a given layer (i) can be fully connected to every other neuron in the next layer (i+1), and neurons in one or more layers may be configured to implement sigmoid or softmax activation functions for classifying each input. Instead of using the classification output of the MLP network, stochastic dropout can be implemented to predict measurement uncertainty. Specifically, a pre-specified percentage of randomly selected nodes within the MLP can be temporarily omitted or ignored during processing. To predict the uncertainty that a given measurement obtained during an exam is correct, multiple feedforward iterations of the model can be run, and during each run, one or more nodes are stochastically dropped from the network. After multiple iterations, variation of the predictions produced by the MLP for a single patient can be used as an indicator of uncertainty. For example, high prediction variation obtained after multiple iterations can indicate high measurement uncertainty, and thus a greater likelihood of measurement error. Likewise, low variation after multiple iterations can indicate low measurement uncertainty. To train the MLP, medical expert annotations of various fetal images and/or measurements, along with the corresponding fetal outcomes, e.g., birth weight, normal birth, abnormal birth, can be used.

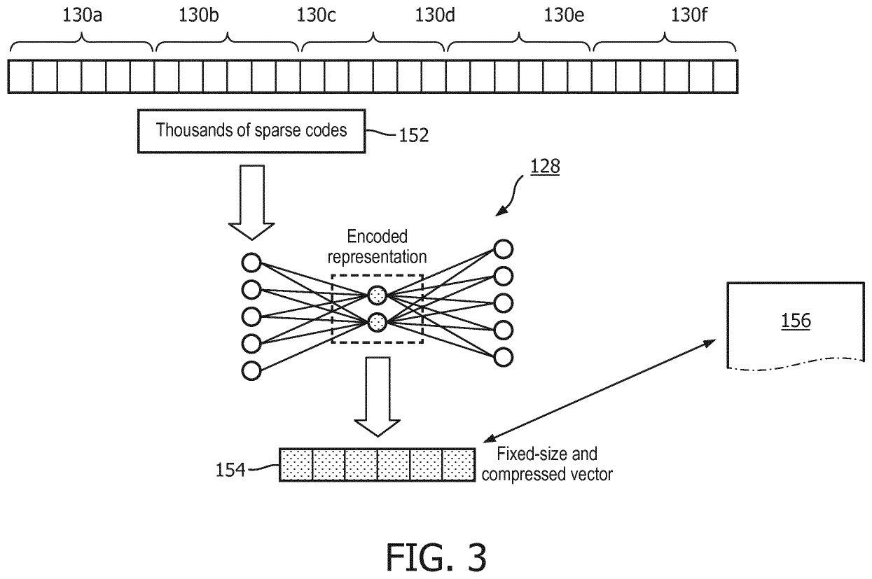

In additional embodiments, unsupervised learning may be implemented via autoencoder-based phenotype stratification and outlier identification. Specifically, autoencoder-based phenotype stratification or Restricted Boltzmann Machine (RBM) can be used to uncover latent structure in the raw input data (embodied in inputs 130) without human input. An example autoencoder-based operation is illustrated in FIG. 3. As shown, neural network 128 can comprise the autoencoder network, which may be configured to receive a plurality, e.g., thousands or more, of sparse codes 152 representative of the various inputs 130 described above. The autoencoder 128 learns to generate a compressed vector 154 of the sparse codes, which may be compared to a set of population-wide training data constituting a manifold 156 of known data points, thereby determining if the combination of new measurement data resembles the training data. If not, the data may be an outlier, which may indicate that a rare anomaly has been detected. The anomaly can embody a real anatomical difference or an incorrect measurement. In either case, the user may be signaled to re-evaluate the outlier to confirm whether the data is, in fact, indicative of an anatomical abnormality, or whether the initial measurement was simply inaccurate. In an example, the compressed vector 154 is processed via a clustering algorithm, such as the t-distributed stochastic neighbor embedding algorithm (t-SNE). According to such an example, the distance of the new measurement data can be compared to the population-based distribution data embodied in the manifold 156 to determine how different the new data is from the training data.

In some examples, after a set of measurements is identified as ambiguous or uncertain, domain expertise in the form of rule-based charts can be applied to evaluate the results. Rule-based charts can be used to identify which measurement(s) appears to be an outlier. In some examples, inaccurate measurements can be identified by iteratively excluding one of the measurements out of a particular data set analyzed via the neural network 128, and then selecting, via the processor 126 or a user, which measurement contributes the most to the measurement uncertainty.

In various embodiments, the neural network 128, image classification network 144 and/or image quality network 148 may be implemented, at least in part, in a computer-readable medium comprising executable instructions executed by a processor, e.g., data processor 126. To train neural network 128, 144 and/or 148, training sets which include multiple instances of input arrays and output classifications may be presented to the training algorithm(s) of the neural network(s) (e.g., AlexNet training algorithm, as described by Krizhevsky, A., Sutskever, I. and Hinton, G. E. “ImageNet Classification with Deep Convolutional Neural Networks,” NIPS 2012 or its descendants).

In some examples, a neural network training algorithm associated with the neural network 128, 144 and/or 148 can be presented with thousands or even millions of training data sets in order to train the neural network to determine a confidence level for each measurement acquired from a particular ultrasound image. In various examples, the number of ultrasound images used to train the neural network(s) may range from about 50,000 to 200,000 or more. The number of images used to train the network(s) may be increased if higher numbers of different anatomical features are to be identified, or to accommodate a greater variety of patient variation, e.g., weight, height, age, etc. The number of training images may differ for different anatomical features, and may depend on variability in the appearance of certain features. Training the network(s) to assess measurement quality associated with features for which population-wide variability is high and may necessitate a greater volume of training images, for example.

The results of an ultrasound scan, including the quality of the obtained measurements embodied in one or more confidence level outputs 132, can be displayed to a user via one or more components of system 100. As shown in FIG. 4, such components can include a display processor 158 communicatively coupled with data processor 126. The display processor 158 is further coupled with a user interface 160, such that the display processor 158 can link the data processor 126 (and thus the one or more neural networks operating thereon) to the user interface 160, enabling the neural network outputs, e.g., measurements and confidence levels, to be displayed on the user interface. In embodiments, the display processor 158 can be configured to generate ultrasound images 162 from the image frames 124 received at the data processor 126. In some examples, the user interface 160 can be configured to display the ultrasound images 162 in real time as an ultrasound scan is being performed, along with one or more notifications 140, which may be overlaid on the images. The notifications 140 can include measurements and associated confidence levels in the form of annotations, color-mapping, percentages, bars, and aural, voice, or haptic rendering, which may be organized in a report 142. Additionally, indications of whether particular measurements satisfy a given threshold can be included in the notifications 140 along with, in some embodiments, one or more instructions for guiding the user to re-acquire a particular measurement.

The user interface 160 can also be configured to receive a user input 166 at any time before, during, or after an ultrasound scan. For instance, the user interface 160 may be interactive, receiving user input 166 indicating confirmation that an anatomical feature has been accurately measured or confirmation that a measurement needs to be reacquired. In some examples, the input 166 may include an instruction to raise or lower threshold 134 or adjust one or more image acquisition settings. As further shown, the user interface 160 can be configured to display a biometry tool widget 123 for acquiring a measurement of an anatomical feature.

The configuration of the components shown in FIG. 4, along with FIG. 1, may vary. For example, the system 100 can be portable or stationary. Various portable devices, e.g., laptops, tablets, smart phones, remote displays and interfaces, or the like, may be used to implement one or more functions of the system 100. Some or all of the data processing may be performed remotely, (e.g., in the cloud). In examples that incorporate such devices, the ultrasound sensor array 112 may be connectable via a USB interface, for example. In some examples, various components shown in FIGS. 1-4 may be combined. For instance, neural network 128 may be merged with the image classification network 144 and/or image quality network 148. According to such embodiments, the output generated by networks 144 and/or 148 may still be input into neural network 128, but the three networks may constitute sub-components of a larger, layered network, for example.

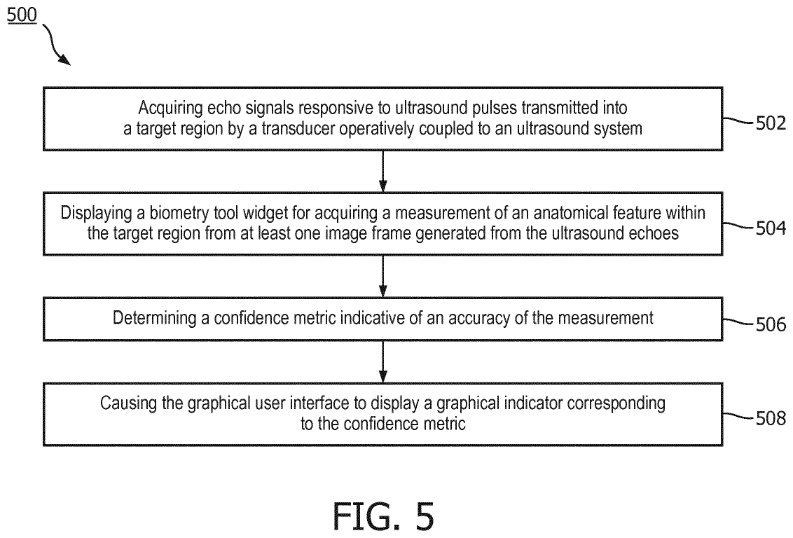

FIG. 5 is a flow diagram of a method of ultrasound imaging performed in accordance with principles of the present disclosure. The example method 500 shows the steps that may be utilized, in any sequence, by the systems and/or apparatuses described herein for determining the quality of one or more anatomical measurements, for example during a fetal scan, which may be performed by a novice user and/or robotic ultrasound apparatus adhering to instructions generated by the system. The method 500 may be performed by an ultrasound imaging system, such as system 100, or other systems including, for example, a mobile system such as LUMIFY by Koninklijke Philips N.V. (“Philips”). Additional example systems may include SPARQ and/or EPIQ, also produced by Philips.

In the embodiment shown, the method 500 begins at block 502 by “acquiring echo signals responsive to ultrasound pulses transmitted into a target region by a transducer operatively coupled to an ultrasound system.”

At block 504, the method involves “displaying a biometry tool widget for acquiring a measurement of an anatomical feature within the target region from at least one image frame generated from the ultrasound echoes.”

At block 506, the method involves “determining a confidence metric indicative of an accuracy of the measurement.”

At block 508, the method involves “causing the graphical user interface to display a graphical indicator corresponding to the confidence metric.”

In various embodiments where components, systems and/or methods are implemented using a programmable device, such as a computer-based system or programmable logic, it should be appreciated that the above-described systems and methods can be implemented using any of various known or later developed programming languages, such as “C”, “C++”, “FORTRAN”, “Pascal”, “VHDL” and the like. Accordingly, various storage media, such as magnetic computer disks, optical disks, electronic memories and the like, can be prepared that can contain information that can direct a device, such as a computer, to implement the above-described systems and/or methods. Once an appropriate device has access to the information and programs contained on the storage media, the storage media can provide the information and programs to the device, thus enabling the device to perform functions of the systems and/or methods described herein. For example, if a computer disk containing appropriate materials, such as a source file, an object file, an executable file or the like, were provided to a computer, the computer could receive the information, appropriately configure itself and perform the functions of the various systems and methods outlined in the diagrams and flowcharts above to implement the various functions. That is, the computer could receive various portions of information from the disk relating to different elements of the above-described systems and/or methods, implement the individual systems and/or methods and coordinate the functions of the individual systems and/or methods described above.

In view of this disclosure it is noted that the various methods and devices described herein can be implemented in hardware, software and firmware. Further, the various methods and parameters are included by way of example only and not in any limiting sense. In view of this disclosure, those of ordinary skill in the art can implement the present teachings in determining their own techniques and needed equipment to affect these techniques, while remaining within the scope of the invention. The functionality of one or more of the processors described herein may be incorporated into a fewer number or a single processing unit (e.g., a CPU) and may be implemented using application specific integrated circuits (ASICs) or general purpose processing circuits which are programmed responsive to executable instruction to perform the functions described herein.

Although the present system may have been described with particular reference to an ultrasound imaging system, it is also envisioned that the present system can be extended to other medical imaging systems where one or more images are obtained in a systematic manner. Accordingly, the present system may be used to obtain and/or record image information related to, but not limited to renal, testicular, breast, ovarian, uterine, thyroid, hepatic, lung, musculoskeletal, splenic, cardiac, arterial and vascular systems, as well as other imaging applications related to ultrasound-guided interventions. Further, the present system may also include one or more programs which may be used with conventional imaging systems so that they may provide features and advantages of the present system. Certain additional advantages and features of this disclosure may be apparent to those skilled in the art upon studying the disclosure, or may be experienced by persons employing the novel system and method of the present disclosure. Another advantage of the present systems and method may be that conventional medical image systems can be easily upgraded to incorporate the features and advantages of the present systems, devices, and methods.

Of course, it is to be appreciated that any one of the examples, embodiments or processes described herein may be combined with one or more other examples, embodiments and/or processes or be separated and/or performed amongst separate devices or device portions in accordance with the present systems, devices and methods.

Finally, the above-discussion is intended to be merely illustrative of the present system and should not be construed as limiting the appended claims to any particular embodiment or group of embodiments. Thus, while the present system has been described in particular detail with reference to exemplary embodiments, it should also be appreciated that numerous modifications and alternative embodiments may be devised by those having ordinary skill in the art without departing from the broader and intended spirit and scope of the present system as set forth in the claims that follow. Accordingly, the specification and drawings are to be regarded in an illustrative manner and are not intended to limit the scope of the appended claims.

1. An ultrasound imaging system comprising:
an ultrasound transducer configured to acquire echo signals responsive to ultrasound pulses transmitted toward a target region of a patient;
a graphical user interface configured to display a biometry tool widget for acquiring a measurement of an anatomical feature within the target region from at least one image frame generated from the ultrasound echoes; and
one or more processors in communication with the ultrasound transducer and configured to:
determine a confidence metric indicative of an accuracy of the measurement; and
cause the graphical user interface to display a graphical indicator corresponding to the confidence metric.

2. The ultrasound imaging system of claim 1, wherein the processors are configured to determine the confidence metric by inputting the at least one image frame into a first neural network trained with imaging data comprising the anatomical feature.

3. The ultrasound imaging system of claim 2, wherein the processors are further configured to determine the confidence metric by inputting a patient statistic, a prior measurement of the anatomical feature, a derived measurement based on the prior measurement, a probability that the image frame contains an anatomical landmark associated with the anatomical feature, a quality level of the image frame, a setting of the ultrasound transducer, or combinations thereof, into the first neural network.

4. The ultrasound imaging system of claim 3, wherein the probability that the image frame contains the anatomical landmark indicates whether a correct imaging plane has been obtained for measuring the anatomical feature.

5. The ultrasound imaging system of claim 1, wherein the graphical user interface is not physically coupled to the ultrasound transducer.

6. The ultrasound imaging system of claim 3, wherein the anatomical feature is a feature associated with a fetus or a uterus, and the derived measurement comprises a gestational age or an age-adjusted risk of a chromosomal abnormality.

7. The ultrasound imaging system of claim 3, wherein the patient statistic comprises a maternal age, a patient weight, a patient height, or combinations thereof.

8. The ultrasound imaging system of claim 3, wherein the quality level of the image frame is based on a distance of the anatomical feature from the ultrasound transducer, an orientation of the biometry tool widget relative to the ultrasound transducer, a distance of a beam focus region to the anatomical feature, a noise estimate obtained via frequency analysis, or combinations thereof.

9. The ultrasound imaging system of claim 1, wherein the processors are further configured to:
apply a threshold to the confidence metric to determine whether the measurement should be re-acquired; and
cause the graphical user interface to display an indication of whether measurement should be re-acquired.

10. The ultrasound imaging system of claim 1, wherein the biometry tool widget comprises a caliper, a trace tool, an ellipse tool, a curve tool, an area tool, a volume tool, or combinations thereof.

11. The ultrasound imaging system of claim 1, wherein the anatomical feature is a feature associated with a fetus or a uterus.

12. The ultrasound imaging system of claim 11, wherein the processors are further configured to determine a gestational age and/or a weight estimate based on the measurement.

13. The ultrasound imaging system of claim 2, wherein the first neural network comprises a multilayer perceptron network configured to perform supervised learning with stochastic dropout, or an autoencoder network configured to generate a compressed representation of the image frame and the measurement, and compare the compressed representation to a manifold of population-based data.

14. A method of ultrasound imaging, the method comprising:
acquiring echo signals responsive to ultrasound pulses transmitted into a target region of a patient by a transducer operatively coupled to an ultrasound system;
displaying a biometry tool widget for acquiring a measurement of an anatomical feature within the target region from at least one image frame generated from the ultrasound echoes;
determining a confidence metric indicative of an accuracy of the measurement; and causing a graphical user interface to display a graphical indicator corresponding to the confidence metric.

15. The method of claim 14, wherein determining the confidence metric comprises inputting the at least one image frame into a first neural network trained with imaging data comprising the anatomical feature.

16. The method of claim 15, further comprising inputting a patient statistic, a prior measurement of the anatomical feature, a derived measurement based on the prior measurement, a probability that the image frame contains an anatomical landmark associated with the anatomical feature, a quality level of the image frame, a setting of the ultrasound transducer, or combinations thereof, into the first neural network.

17. The method of claim 16, wherein the patient statistic comprises a maternal age, a patient weight, a patient height, or combinations thereof.

18. The method of claim 16, wherein the anatomical feature is a feature associated with a fetus or a uterus, and the derived measurement comprises a gestational age or an age-adjusted risk of a chromosomal abnormality.

19. The method of claim 14, the anatomical feature is a feature associated with a fetus or a uterus, and further comprises determining a gestational age and/or a weight estimate based on the measurement.

20. A non-transitory computer-readable medium comprising executable instructions, which when executed cause a processor of a medical imaging system to perform the methods of claim 14.
